(19) United States
(12) Patent Application Publication
(10) Pub. No.: US 2022/0130543 A1
(43) Pub. Date: Apr. 28, 2022

(54) MEDICAL INFORMATION PROCESSING APPARATUS

(71) Applicant: CANON MEDICAL SYSTEMS CORPORATION, Otawara-shi (JP)

(72) Inventors: Kazumasa NORO, Shioya-gun (JP); Yusuke KANO, Nasushiobara (JP); Anri SATO, Nasushiobara (JP); Minoru NAKATSUGAWA, Yokohama (JP)

(73) Assignee: CANON MEDICAL SYSTEMS CORPORATION, Otawara-shi (JP)

(21) Appl. No.: 17/504,894

(22) Filed: Oct. 19, 2021

(30) Foreign Application Priority Data
Oct. 28, 2020 (JP) 2020-180618

(51) Int. Cl. G16H 50/20; G16H 15/00

(52) U.S. Cl. CPC G16H 50/20; G16H 15/00

(57) ABSTRACT

A medical information processing apparatus according to an embodiment includes processing circuitry. The processing circuitry acquires a first diagnostic result by a clinical decision support system before learning of first data, a second diagnostic result by the clinical decision support system after learning of the first data, a diagnostic result by a doctor, and the first data. The processing circuitry extracts an example that is subjected to an influence of updating of the clinical decision support system by a prescribed criterion or more as an example to be presented. The processing circuitry outputs the example to be presented in association with a diagnosis influence degree indicator indicating a degree of an influence that the clinical decision support system exerts on diagnosis by the doctor.

### CROSS-REFERENCE TO RELATED APPLICATIONS

This application is based upon and claims the benefit of priority from Japanese Patent Application No. 2020-180618, filed on Oct. 28, 2020; the entire contents of which are incorporated herein by reference.

### FIELD

Embodiments described herein relate generally to a medical information processing apparatus.

### BACKGROUND

Conventionally a technique has been known of a clinical decision support system (CDSS) to support doctors in their diagnosis of patients. In the CDSS in general, a learned model presenting information on a disease of the patient and a method for treating the disease based on results obtained by learning of learning data based on past records by machine learning or the like is used. In the CDSS, the model may be updated by performing learning based on new learning data after the start of operation as well.

### BRIEF DESCRIPTION OF THE DRAWINGS

FIG. 1 is a diagram of an example of an entire configuration of an information processing system according to a first embodiment;

FIG. 2 is a diagram of an example of a decision tree for use in a clinical decision support system (CDSS) according to the first embodiment;

FIG. 3 is a diagram of an example of a change in output before and after updating of the CDSS according to the first embodiment;

FIG. 4 is a diagram of an example of information that the medical information processing system according to the first embodiment acquires;

FIG. 5 is a diagram of an example of a relation between a diagnostic result by the CDSS and a diagnostic result by a doctor according to the first embodiment;

FIG. 6 is a diagram of another example of the relation between the diagnostic result by the CDSS and the diagnostic result by the doctor according to the first embodiment;

FIG. 7 is a diagram of still another example of the relation between the diagnostic result by the CDSS and the diagnostic result by the doctor according to the first embodiment;

FIG. 8 is diagram of an example of a method for extracting influential variables according to the first embodiment;

FIG. 9 is a diagram of an example of an analytical screen according to the first embodiment;

FIG. 10 is a diagram of an example of addition of new examples according to the first embodiment;

FIG. 11 is diagram of an example of classification of variables according to the first embodiment;

FIG. 12 is a diagram of an example of learning processing according to the first embodiment;

FIG. 13 is a flowchart of an example of a procedure of updating support processing according to the first embodiment;

FIG. 14 is a diagram of an example of the analytical screen according to a second embodiment; and

FIG. 15 is a diagram of an example of the analytical screen according to a first modification.

### DETAILED DESCRIPTION

The following describes embodiments of a medical information processing apparatus in detail with reference to the accompanying drawings.

### First Embodiment

A medical information processing apparatus according to an embodiment includes processing circuitry. The processing circuitry acquires a first diagnostic result for each of a plurality of patients having undergone diagnosis by a clinical decision support system before learning of first data, a second diagnostic result for each of the patients having undergone diagnosis by the clinical decision support system after learning of the first data, a diagnostic result by a doctor for each of the patients having undergone diagnosis, and the first data. The processing circuitry extracts an example that is subjected to an influence of updating of the clinical decision support system by learning of the first data by a first prescribed criterion or more among a plurality of examples included in the first data as an example to be presented based on the first diagnostic result, the second diagnostic result, and the diagnostic result by the doctor. The processing circuitry outputs the example to be presented in association with a diagnosis influence degree indicator indicating a degree of an influence that the clinical decision support system exerts on diagnosis by the doctor in the example to be presented. The first data includes the examples in which disease-related information for each of the patients having undergone diagnosis and a true value representing a treatment result for each of the patients having undergone diagnosis are associated with each other.

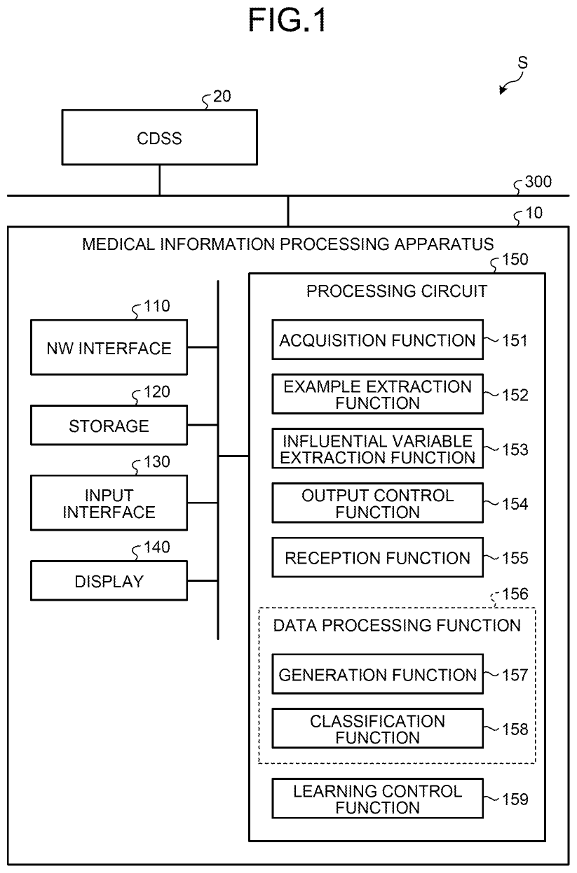

FIG. 1 is a diagram of an example of an entire configuration of an information processing system S according to a first embodiment. As illustrated in FIG. 1, the information processing system S includes a medical information processing apparatus 10 and a clinical decision support system (CDSS) 20. The medical information processing apparatus 10 and the CDSS 20 are installed in a medical institution such as a hospital, for example. The medical information processing apparatus 10 is connected to the CDSS 20 in a communicable manner via a network 300 such as a hospital local area network (LAN). An electronic medical record system or the like (not illustrated) may further be connected to the medical information processing apparatus 10 and the CDSS 20.

The CDSS 20 provides information useful when a medical practitioner such as a doctor makes a clinical judgment. In the present embodiment, the CDSS 20 presents a diagnostic result of a patient based on results obtained by learning of learning data based on past records by deep learning or another machine learning to support the doctor. More specifically, the CDSS 20 of the present embodiment outputs a method of treatment estimated to be effective against a disease from which the patient has and a treatment result estimated when the method of treatment is conducted.

The CDSS 20 includes a learned model that has learned learning data. The learned model is a learned model generated by deep learning such as a neural network, for example. Examples of methods of deep learning include, but are not limited to, a decision tree, a convolutional neural network (CNN), and a recurrent neural network (RNN).

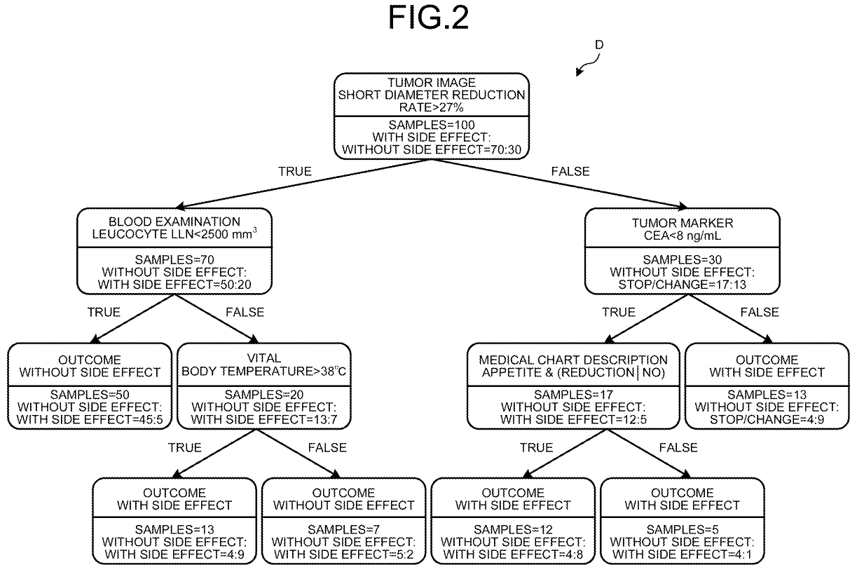

FIG. 2 is a diagram of an example of a decision tree D for use in the CDSS 20 according to the first embodiment. As illustrated in FIG. 2, the decision tree D estimates an outcome based on diseases-related information of the patient such as a medical image of the patient, a blood examination result, a tumor marker, vital data, findings by the doctor described in an electronic medical chart, and a blood examination result for example. The outcome of the decision tree D illustrated in FIG. 2 indicates occurrence of a side effect to the patient. The learned model for use in the CDSS 20 may employ either method of recurrence or classification. The outcome output by the CDSS 20, that is, a diagnostic result may be not only classification such as the occurrence of a side effect but also a value such as a side effect risk score estimated by a method of recurrence.

The CDSS 20 includes a server apparatus or a normal computer such as a personal computer (PC), for example.

The CDSS 20 learns new learning data after the start of operation as well and can thereby improve accuracy. The improvement in accuracy is making the estimated treatment result output by the CDSS 20 close to an actual treatment result of the patient. In the present embodiment, the CDSS 20 learning the new learning data is referred to as updating the CDSS 20. The new learning data is an example of the first data of the present embodiment.

The CDSS 20 after updating may output a different result from that of the CDSS 20 before updating. It is ideal that updating of the CDSS 20 improves the accuracy of the CDSS 20 for all examples, but in actuality, it may be difficult to improve diagnostic accuracy uniformly for all the examples. Given this, in the present embodiment, the medical information processing apparatus 10 described below improves accuracy preferentially for an example having a high degree of influence that the CDSS 20 exerts on the diagnosis by the doctor.

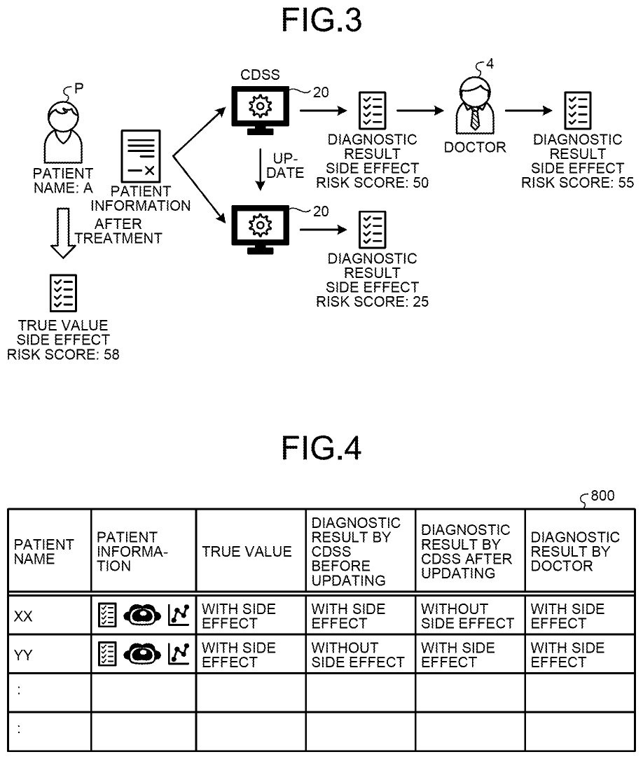

FIG. 3 is a diagram of an example of a change in output before and after updating of the CDSS 20 according to the first embodiment. The example illustrated in FIG. 3 illustrates the CDSS 20 before learning the new learning data on the upper row and the CDSS 20 after leaning the new learning data on the lower row. The “CDSS 20 before learning the new learning data” may be a CDSS that has repeated a plurality of number of times of learning in the past or be a CDSS that performed learning before the start of operation and has not yet performed any learning after the start operation.

In the example illustrated in FIG. 3, when receiving input of patient information of a patient P, the CDSS 20 before updating and the CDSS 20 after updating output a diagnostic result of the patient P.

Specifically, the CDSS 20 outputs a method of treatment that is estimated to be effective against a disease that the patient P has and a treatment result estimated when the method of treatment is conducted. The CDSS 20 outputs the type and the dose of a medicine to be administered as the method of treatment, for example. The CDSS 20 outputs the side effect risk score when the medicine is administered as the treatment result. The side effect risk score is a value representing the intensity of a side effect occurring by administration, for example. The information included in the diagnostic result is not limited to these pieces of information.

The patient information includes disease-related information of the patient P and attribute information of the patient P.

Examples of the attribute information of the patient P include, but are not limited to, the name, age, gender, height, and weight of the patient P. Not the name, but the identification (ID) of the patient P may be used.

The disease-related information of the patient P includes a plurality of kinds of examination results on the patient P. Examples of the kinds of examination results include vital data such as body temperature and blood pressure, blood examination results such as a leukocyte count, a tumor marker, and an image measured value such as the size of a tumor. In the present embodiment, the examination results include not only quantitative examination results but also findings by the doctor or a nurse such as having appetite or reduction in appetite. The findings may be included in the disease-related information of the patient P as information different from the examination results. The disease-related information of the patient P can include a disease name.

In the example illustrated in FIG. 3, the CDSS 20 before updating outputs a side effect risk score “50” as the diagnostic result. The diagnostic result output by the CDSS 20 before updating is an example of the first diagnostic result of the present embodiment.

In FIG. 3, not only the CDSS 20, but also a doctor 4 performs diagnosis on the patient P. In the diagnostic result by the doctor 4, the side effect risk score is “55.” It is assumed that in the example illustrated in FIG. 3 the doctor 4 performs diagnosis in view of his/her own experiences and other information with reference to the diagnostic result output from the CDSS 20 before updating. The doctor 4 is an example of a user according to the present embodiment.

Even when the CDSS 20 is used, it is general that the doctor 4 makes a final diagnosis. Thus, in the present embodiment, it is assumed that the diagnostic result by the doctor 4 has been given for the patient P having undergone diagnosis.

The “true value” illustrated in FIG. 3 indicates an actual treatment result of the patient P. The diagnostic results by the CDSS 20 and the doctor 4 are estimated values before treatment is actually conducted, whereas the “true value” is a treatment result revealed by an examination or the like after the treatment has been conducted. In this example, it is assumed that the intensity of the actual side effect of the patient P was about a side effect risk score “58.”

In the example illustrated in FIG. 3, the CDSS 20 after updating, even receiving input of the same patient information as that before updating, outputs a side effect risk score “25,” which is different from that before updating. The diagnostic result output by the CDSS 20 after updating is an example of the second diagnostic result of the present embodiment.

The difference between the side effect risk score “50” output by the CDSS 20 before updating and the side effect risk score “58” of the true value was “8,” whereas the difference between the side effect risk score “25” output by the CDSS 20 after updating and the side effect risk score “58” of the true value was “33.” In this case, the difference between the diagnostic result by the CDSS 20 after updating and the true value is larger than the difference between the diagnostic result by the CDSS 20 before updating and the true value.

In such a case, when the doctor 4 performs diagnosis on a patient similar to the patient P after the CDSS 20 has been updated, when the diagnostic result by the CDSS 20 is referred to, there is a possibility that a judgment by the doctor 4 will be guided to a direction separating from the true value by the CDSS 20 after updating.

The difference between the first diagnostic result output by the CDSS 20 before updating and the true value is an example of a first difference of the present embodiment. The difference between the second diagnostic result output by the CDSS 20 after updating and the true value is an example of a second difference of the present embodiment.

Referring back to FIG. 1, the medical information processing apparatus 10 performs a simulation of update, processing on the learning data, and the like before the CDSS 20 learns the new learning data. The medical information processing apparatus 10 is also called a CDSS update support apparatus, for example.

The medical information processing apparatus 10 is a server apparatus or a personal computer (PC), for example, and includes a network (NW) interface 110, a storage 120, an input interface 130, a display 140, and a processing circuit 150.

The NW interface 110 is connected to the processing circuit 150 and controls transmission and communication of various kinds of data performed between the medical information processing apparatus 10 and the CDSS 20. The NW interface 110 is implemented by a network card, a network adapter, a network interface controller (NIC), or the like.

The storage 120 stores therein various kinds of information for use in the processing circuit 150 in advance. The storage 120 stores therein various kinds of computer programs.

The input interface 130 is implemented by a trackball, switch button, a mouse, a keyboard, a touch pad, which performs input operations through touching onto an operating face, a touch screen, in which a display screen and a touch pad are integrated with each other, a noncontact input circuit including an optical sensor, a voice input circuit, or the like. The input interface 130 is connected to the processing circuit 150 and converts an input operation received from an operator to an electric signal and outputs the electric signal to the processing circuit 150. In the present specification, the input interface is not limited to one including a physical operating part such as a mouse or a keyboard. Examples of the input interface 130 include an electric signal processing circuit receiving an electric signal corresponding to an input operation from an external input device provided separately from the apparatus and outputting this electric signal to the processing circuit 150.

The display 140 is a liquid crystal display, an organic electro-luminescence (OEL) display, or the like. The input interface 130 and the display 140 may be integrated with each other. The input interface 130 and the display 140 may be implemented by a touch panel, for example. The display 140 is an example of an output unit of the present embodiment. Although the present embodiment describes a case in which the display 140 is included in the medical information processing apparatus 10, the display 140 may be provided outside the medical information processing apparatus 10.

The processing circuit 150 is processing circuitry reading the computer programs from the storage 120 and executing them to implement functions corresponding to the respective computer programs. The processing circuit 150 of the present embodiment includes an acquisition function 151, an example extraction function 152, an influential variable extraction function 153, an output control function 154, a reception function 155, a data processing function 156, a generation function 157, a classification function 158, and a learning control function 159. The acquisition function 151 is an example of an acquisition unit. The example extraction function 152 is an example of an example extraction unit. The influential variable extraction function 153 is an example of an influential variable extraction unit. The output control function 154 is an example of an output controller. The reception function 155 is an example of a receiver. The data processing function 156 is an example of a data processing unit or a converter. The generation function 157 is an example of a generator. The classification function 158 is an example of a classification unit. The learning control function 159 is an example of a learning controller.

The processing functions of the acquisition function 151, the example extraction function 152, the influential variable extraction function 153, the output control function 154, the reception function 155, the data processing function 156, the generation function 157, the classification function 158, and the learning control function 159 as the components of the processing circuit 150 are stored in the storage 120 in the form of a computer-executable computer program, for example. The processing circuit 150 is the processing circuitry. The processing circuit 150 reads the computer programs from the storage 120 and executes them to implement the functions corresponding to the respective computer programs, for example. In other words, the processing circuit 150 having read the computer programs has the respective functions indicated within the processing circuit 150 in FIG. 1. Although it has been described that in FIG. 1 the single processing circuitry implements the processing functions performed by the acquisition function 151, the example extraction function 152, the influential variable extraction function 153, the output control function 154, the reception function 155, the data processing function 156, the generation function 157, the classification function 158, and the learning control function 159, a plurality of independent pieces of processing circuitry may be combined to form the processing circuit 150, and the pieces of processing circuitry may execute the respective computer programs to implement the functions. Although it has been described that in FIG. 1 the single storage 120 stores therein the computer programs corresponding to the respective processing functions, a plurality of storage may be placed in a distributed manner, and the processing circuit 150 may read corresponding computer programs from the individual storage.

Although the above description describes an example in which the “processing circuitry” reads the computer programs corresponding to the respective functions from the storage and executes them, the embodiment is not limited to this example. The term “processing circuitry” means a circuit such as a central processing unit (CPU), a graphics processing unit (GPU), an application specific integrated circuit (ASIC), a programmable logic device (a simple programmable logic device (SPLD) or a complex programmable logic device (CPLD), for example), or a field programmable gate array (FPGA), for example. When the processing circuitry is the CPU, for example, the processing circuitry reads the computer programs stored in the storage and executes them to implement the functions. When the processing circuitry is the ASIC, in place of storing the computer programs in the storage 120, the functions are directly incorporated as logical circuits into the circuit of the processing circuitry. Each piece of processing circuitry of the present embodiment is not limited to a case in which each piece of processing circuitry is formed as a single circuit, and a plurality of independent circuits may be combined to form one piece of processing circuitry and to implement the functions. Furthermore, the components in FIG. 1 may be integrated into one piece of processing circuitry to implement the functions.

The acquisition function 151 acquires the first diagnostic result and the second diagnostic result for each of a plurality of patients P having undergone diagnosis and a diagnostic result by the doctor 4 for each of the patients P having undergone diagnosis supported by the CDSS 20 before updating from the CDSS 20. The acquisition function 151 acquires the new learning data from the CDSS 20.

The new learning data is an example of the first data and is learning data slated to be used for the updating of the CDSS 20. In this case, the first diagnostic result is a diagnostic result by the CDSS 20 before learning of the new learning data. The second diagnostic result is a diagnostic result by the CDSS 20 after learning of the new learning data.

At a point in time of this acquisition, updating of the CDSS 20 has not been finalized. The second diagnostic result is not an actual updated result but a simulation result when the CDSS 20 is updated, for example. The acquisition function 151 may acquire the second diagnostic result from an information processing apparatus other than the CDSS 20, or an information processing apparatus dedicated to simulations, for example.

The new learning data of the present embodiment includes a plurality of examples in which the disease-related information for each of the patients P having undergone diagnosis and the true value representing the treatment result for each of the patients having undergone diagnosis are associated with each other.

As illustrated in FIG. 3, the disease-related information includes the kinds of examination results on the patient P. The example is not necessarily by the patient P; when one patient P has undergone diagnosis a plurality of times for a plurality of diseases, they may be included in the new learning data as separate examples. As described above, the disease-related information includes the attribute information of each of the patients having undergone diagnosis.

FIG. 4 is a diagram of an example of information that the medical information processing apparatus 10 according to the first embodiment acquires. One record of acquired data 800 illustrated in FIG. 4 corresponds to one example.

As illustrated in FIG. 4, the patient information of each of the patients P having undergone diagnosis, the true value, the diagnostic result by the CDSS 20 before updating (the first diagnostic result), the diagnostic result by the CDSS 20 after updating (the second diagnostic result), and the diagnostic result by the doctor 4 are associated with each other for each example. Although FIG. 4 gives illustration in the form of a table for the sake of description, the pieces of information included in the acquired data 800 are only required to be associated with each other for each example and are not necessarily formed as one table.

The acquisition function 151 transmits the acquired new learning data, the first diagnostic result, the second diagnostic result, and the diagnostic result by the doctor 4 for each of the patients P having undergone diagnosis supported by the CDSS 20 before updating to the example extraction function 152.

Referring back to FIG. 1, the example extraction function 152 extracts an example that is subjected to an influence of updating of the CDSS 20 by learning of the new learning data by the first prescribed criterion or more among the examples included in the new learning data as the example to be presented based on the first diagnostic result, the second diagnostic result, and the diagnostic result by the doctor 4 for each of the patients P having undergone diagnosis supported by the CDSS 20 before updating. The example to be presented indicates an example to be displayed on an analytical screen described below. In the present embodiment, determining any example as the example to be presented out of the examples by the example extraction function 152 is referred to as extracting the example to be presented.

In the present embodiment, the example that is subjected to the influence of updating of the CDSS 20 by the first prescribed criterion or more refers to an example in which the second diagnostic result by the CDSS 20 after updating further separates from the true value than the first diagnostic result by the CDSS 20 before being updated.

In the present embodiment, the difference between the first difference as the difference between the first diagnostic result and the true value and the second difference as the difference between the second diagnostic result and the true value is an example of an updating influence degree indicator. More specifically, the updating influence degree indicator is a value obtained by subtracting the first difference from the second difference. In this case, the second difference larger than the first difference, that is, the diagnostic result further separating from the true value by updating gives a larger updating influence degree indicator.

In the present embodiment, the example extraction function 152 further makes it a condition for the example to be presented that the fact that an influence exerted on the diagnostic result by the doctor 4 is larger than a third prescribed criterion. In the present embodiment, the example extraction function 152 determines that when a degree of similarity between the first diagnostic result and the diagnostic result by the doctor 4 for each of the patients P having undergone diagnosis supported by the CDSS 20 before updating to be an example is higher, influence exerted on the diagnostic result by the doctor 4 is larger. Thus, the example extraction function 152 makes it a condition for the example to be presented that the fact that the difference between the first diagnostic result and the diagnostic result by the doctor 4 supported by the CDSS 20 is a second prescribed criterion or less. It is assumed that when the difference between the first diagnostic result and the diagnostic result by the doctor 4 is the second prescribed criterion or less, the influence exerted on the diagnostic result by the doctor 4 is larger than the third prescribed criterion.

The difference between the first diagnostic result and the diagnostic result by the doctor 4 supported by the CDSS 20 is an example of the diagnosis influence degree indicator of the present embodiment. It is assumed that as the difference becomes smaller between the first diagnostic result and the diagnostic result by the doctor 4 supported by the CDSS 20, the diagnosis influence degree indicator becomes larger.

That is to say, the example extraction function 152 determines an example in which the first difference is the second difference or less and the difference between the first diagnostic result and the diagnostic result by the doctor 4 supported by the CDSS 20 is the second prescribed criterion or less among the examples included in the new learning data to be the example to be presented. The second prescribed criterion is a certain threshold, for example. More specifically, the example extraction function 152 extracts an example satisfying both Expression (1) and Expression (2) as the example to be presented.

|FIRST DIAGNOSTIC RESULT BY CDSS BEFORE UPDATING−DIAGNOSTIC RESULT BY DOCTOR|≤THRESHOLD (1)

|FIRST DIAGNOSTIC RESULT BY CDSS BEFORE UPDATING−TRUE VALUE|≤|SECOND DIAGNOSTIC RESULT BY CDSS AFTER UPDATING−TRUE VALUE| (2)

Expression (1) prescribes that the absolute value of the value obtained by subtracting the diagnostic result by the doctor 4 from the first diagnostic result by the CDSS 20 before updating is the threshold or less. Expression (2) prescribes that the absolute value of the value obtained by subtracting the true value from the first diagnostic result by the CDSS 20 before updating is the absolute value of the value obtained by subtracting the true value from the second diagnostic result by the CDSS 20 after updating or less.

The threshold of Expression (1) may be set by the user such as the doctor 4 or be the average, the median, or the like of the absolute value of the result of subtraction of the diagnostic result by the doctor 4 from the first diagnostic result by the CDSS 20 before updating. The extraction condition for the example to be presented is not limited to Expression (1) or Expression (2).

When the diagnostic results are not represented by values, a condition other than the threshold may be used as the second prescribed criterion. When the diagnostic results are output in a binary manner, or “present” or “absent,” such as the occurrence of a side effect, for example, “the absence of the difference between the first diagnostic result and the diagnostic result by the doctor 4 supported by the CDSS 20” may be the second prescribed criterion. In this case, if the difference between the first diagnostic result and the diagnostic result by the doctor 4 supported by the CDSS 20 is absent, the absence is equal to the second prescribed criterion. In this case as well, “the difference is the second prescribed criterion or less,” and thus the condition that “the difference between the first diagnostic result and the diagnostic result by the doctor 4 supported by the CDSS 20 is the prescribed criterion or less” is satisfied. Alternatively, the example extraction function 152 may convert a non-numerical diagnostic result into a value to compute the difference.

The patient P satisfying Expression (2) is, in other words, the patient P for which the accuracy of the diagnostic result reduces before and after updating of the CDSS 20. In such a patient P, an influence to which the diagnostic result by the CDSS 20 is subjected by updating is large.

As exemplified in Expression (1), the subject P for which the difference between the first diagnostic result and the diagnostic result by the doctor 4 supported by the CDSS 20 is the second prescribed criterion or less is, in other words, the subject P for which the diagnostic result given by the doctor 4 and the first diagnostic result resemble each other. In the present embodiment, it is assumed that when the first diagnostic result and the diagnostic result by the doctor 4 supported by the CDSS 20 resemble each other, there is a possibility that in the diagnosis on the patient P the doctor 4 has been influenced by the diagnostic result by the CDSS 20.

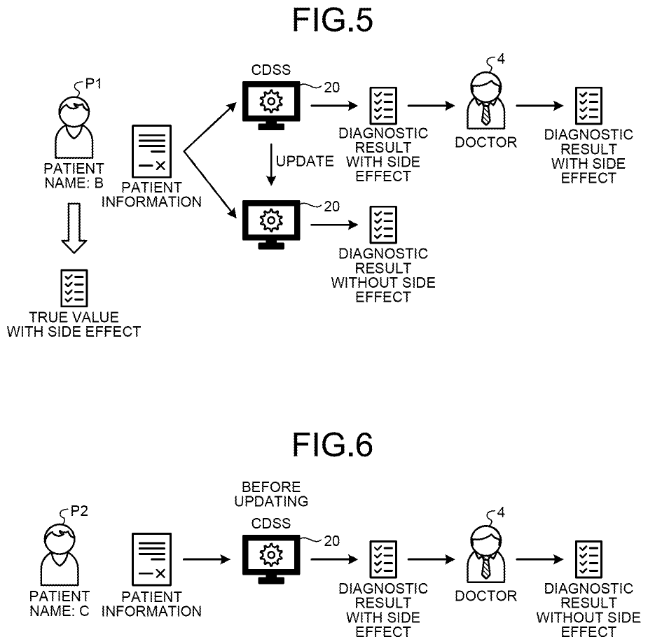

The following describes the influence that the CDSS 20 exerts on the diagnostic result by the doctor 4 with reference to FIG. 5 to FIG. 7. Patients P1 to P3 having undergone diagnosis illustrated in FIG. 5 to FIG. 7 are assumed to be different people. When the individuals of the patients P1 to P3 having undergone diagnosis are not discriminated from each other in particular, they are referred to simply as the patient P.

FIG. 5 is a diagram of an example of a relation between the diagnostic result by the CDSS 20 and the diagnostic result by the doctor 4 according to the first embodiment.

In the example illustrated in FIG. 5, the CDSS 20 before updating has output the first diagnostic result indicating that a side effect occurs in the patient P1. The doctor 4 has also given a diagnostic result indicating that a side effect occurs in the patient P1 like the CDSS 20 before updating. If the second prescribed criterion is “that the first diagnostic result and the diagnostic result by the doctor 4 are the same,” that is, the threshold of the difference is “0,” the difference between the first diagnostic result and the diagnostic result by the doctor 4 supported by the CDSS 20 for the patient P1 is the second prescribed criterion or less.

In this case, there is a high possibility that the doctor 4 has performed diagnosis with reference to the first diagnostic result by the CDSS 20 before updating. When the other patient P having examination results similar to those of the patient P1 undergoes diagnosis, for example, there is a possibility that the doctor 4 performs diagnosis under the influence of the diagnostic result by the CDSS 20 like the example of the patient P1.

Meanwhile, the treatment result, that is, the true value of the patient P1 having undergone treatment was “side effect present.” in this case, the first diagnostic result is equal to the true value. On the other hand, the second diagnostic result by the CDSS 20 after updating, which is “side effect absent,” is different from the true value. In this case, the first difference is smaller than the second difference, which satisfies Expression (2).

In the patient P1 illustrated in FIG. 5, the first difference is the second difference or less and the difference between the first diagnostic result and the diagnostic result by the doctor 4 supported by the CDSS 20 is the second prescribed criterion or less, and thus the example extraction function 152 extracts the example of the patient P1 as the example to be presented.

FIG. 6 is a diagram of another example of the relation between the diagnostic result by the CDSS 20 and the diagnostic result by the doctor 4 according to the first embodiment. In the example illustrated in FIG. 6, the doctor 4 has given a diagnostic result different from the first diagnostic result by the CDSS 20 before updating. If the second prescribed criterion is “that the first diagnostic result and the diagnostic result by the doctor 4 are the same,” that is, the threshold of the difference is “0,” the example of the patient P2 does not satisfy the criterion. As illustrated in FIG. 6, when the difference between the diagnostic result by the CDSS 20 before updating and the diagnostic result by the doctor 4 is large, it is estimated in general that the influence that the CDSS 20 before updating has exerted on the doctor 4 is small. In this case, the example extraction function 152 does not extract the example of the patient P2 as the example to be presented.

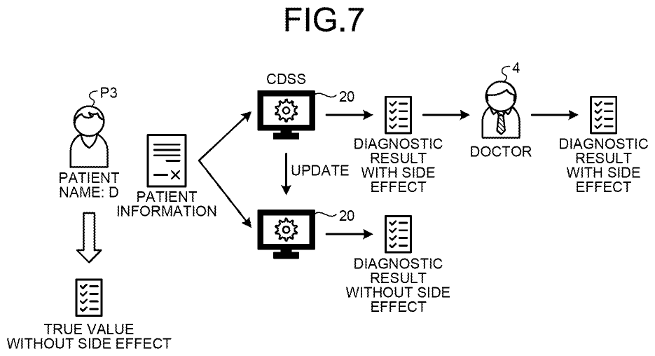

FIG. 7 is a diagram of still another example of the relation between the diagnostic result by the CDSS 20 and the diagnostic result by the doctor 4 according to the first embodiment. In the example illustrated in FIG. 7, the first diagnostic result by the CDSS 20 before updating for the patient P3 is “side effect present,” the second diagnostic result by the CDSS 20 after updating is “side effect absent,” and the true value is “side effect absent.” That is to say, in the example illustrated in FIG. 7, the accuracy of diagnosis for the patient P3 by the CDSS 20 has increased by updating. In this case, the first difference is larger than the second difference, which does not satisfy Expression (2). In this case, the example extraction function 152 does not extract the example of the patient P3 as the example to be presented.

Although FIG. 5 to FIG. 7 illustrate examples in which the same doctor 4 has performed diagnosis, different doctors 4 may perform diagnosis.

The example extraction function 152 sends out the new learning data, the first diagnostic result, the second diagnostic result, and the diagnostic result by the doctor 4 for each of the patients P having undergone diagnosis supported by the CDSS 20 before updating corresponding to the extracted example to be presented to the output control function 154. The example extraction function 152 sends out the diagnosis influence degree indicator and the updating influence degree indicator corresponding to the extracted example to be presented to the output control function 154.

Referring back to FIG. 1, the influential variable extraction function 153 extracts an item having influenced a change in the diagnostic result by the CDSS 20 before and after updating among the kinds of examination results for each of the patients P having undergone diagnosis included in the new learning data as an influential variable. In the present embodiment, the influential variable is fixed by the classification function 158 described below, and thus more accurately, the influential variable extraction function 153 extracts an “influential variable candidate.” in the following, the influential variable candidate is also referred to as the influential variable except a case in which it is described in a discriminated manner in Particular.

In the present embodiment, each item included in the examination results is referred to as a variable. In other word, the influential variable extraction function 153 estimates, when the first diagnostic result and the second diagnostic result by the CDSS 20 are different from each other, through what variable's influence the change has occurred. The influential variable extraction function 153 extracts the variable having influenced the change in the diagnostic result by the CDSS 20 as the influential variable.

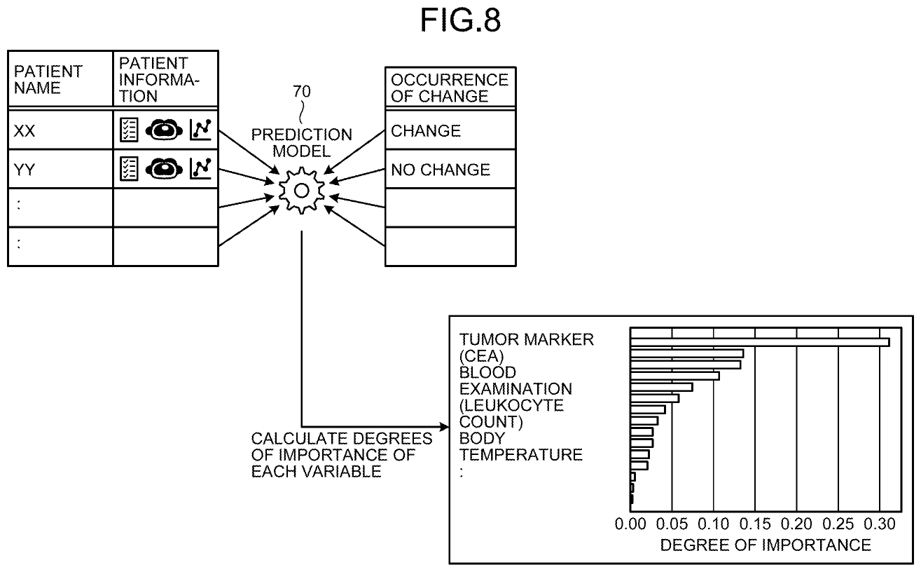

FIG. 8 is a diagram of an example of a method for extracting the influential variable according to the first embodiment. As illustrated in FIG. 8, the influential variable extraction function 153 inputs the patient information for each example included in the new learning data and the occurrence of the change in the diagnostic result by the CDSS 20 before and after updating for each example to a prediction model 70.

The occurrence of the change is also referred to as a label for the patient information. The influential variable extraction function 153 associates a label of “change” (or “change example”) with an example for which there has been a change in the diagnostic result by the CDSS 20 before and after updating, associates a label of “no change” (or “no change example”) with an example for which there has been no change in the diagnostic result by the CDSS 20 before and after updating, and inputs the examples to the prediction model 70. Each example included in the new learning data is identified as the change example or the no change example based on a difference between the first diagnostic result and the diagnostic result by the doctor supported by the CDSS 20 and a difference between the first diagnostic result and the second diagnostic result. For example, the example for which there has been a change in the diagnostic result by the CDSS 20 before and after updating is also referred to as a “change example,” whereas the example for which there has been no change in the diagnostic result by the CDSS 20 before and after updating is also referred to as a “no change example.”

More specifically, the occurrence of the change in the diagnostic result by the CDSS 20 before and after updating is the occurrence of the difference between the first diagnostic result and the second diagnostic result. When the first diagnostic result and the second diagnostic result are represented by values, the influential variable extraction function 153 may determine a case to be “no change” when the difference between the first diagnostic result and the second diagnostic result is a prescribed threshold or less and determine a case to be “change” when the difference between the first diagnostic result and the second diagnostic result is larger than the prescribed threshold.

As described above, the examination results include the vital data such as the body temperature and the blood pressure of the patient P, the blood examination results such as a leukocyte count, the tumor marker, the image measured value such as the size of a tumor, the findings by the doctor or the nurse, and the like, for example. The prediction model 70 outputs the magnitude of an influence that each item of these examination results, that is, each variable exerts on the change in the diagnostic result by the CDSS 20 before and after updating. In the present embodiment, the magnitude of the influence that each variable exerts on the change in the diagnostic result by the CDSS 20 before and after updating is referred to as a degree of importance of each variable. In the example illustrated in FIG. 8, the prediction model 70 represents the degree of importance of each variable with a decimal between 0 and 1. A larger value means that the variable exerts a larger influence on the change in the diagnostic result by the CDSS 20 before and after updating.

The prediction model 70 is assumed to be a learned model generated by machine learning such as random forest, for example. The method of machine learning is not limited to this example, and other known methods of machine learning can be employed. The prediction model 70 may be stored in the storage 120 or be incorporated into the influential variable extraction function 153.

In the present embodiment, the influential variable extraction function 153 determines a variable for which the degree of importance is as large as being a prescribed criterion or more to be the influential variable.

Determining any variable to be the influential variable out of a plurality of variables by the influential variable extraction function 153 is referred to as extracting the influential variable.

The influential variable extraction function 153 sends out the extracted influential variable and the degree of importance of the influential variable to the output control function 154.

Referring back to FIG. 1, the output control function 154 causes the display 140 to output the example to be presented extracted by the example extraction unit 152 in association with the diagnosis influence degree indicator. In the present embodiment, a screen on which the example to be presented and the diagnosis influence degree indicator are displayed is called the analytical screen. In the present embodiment, “output” includes “display.” The method of output is not limited to displaying. The output control function 154 may output the example to be presented to an information processing apparatus other than the medical information processing apparatus 10 in association with the diagnosis influence degree indicator, for example.

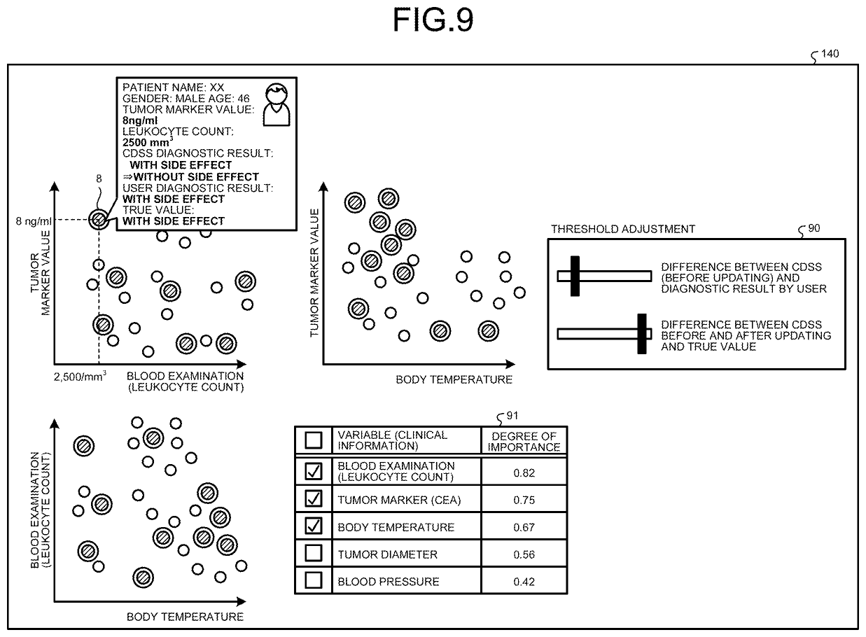

FIG. 9 is a diagram of an example of the analytical screen according to the first embodiment. As illustrated in FIG. 9, the output control function 154 causes the analytical screen on the display 140 to display a list 91 of influential variables extracted by the influential variable extraction function 153. The list 91 includes the name and the degree of importance of the variables as an example. The user can select any influential variable as an object to be analyzed from the list 91. In the example illustrated in FIG. 9, the user has selected three influential variables: “leukocyte count,” “tumor marker,” and “body temperature.”

The output control function 154 causes the display 140 to output the example to be presented in association with the influential variable. More specifically, the output control function 154 causes the analytical screen to display a graph with two each of the influential variables selected by the user as axes. The example illustrated in FIG. 9 displays three kinds of graphs: a graph with “tumor marker” and “leukocyte count” as the axes, a graph with “tumor marker” and “body temperature” as the axes, and a graph with “leukocyte count” and “body temperature” as the axes. Although in the example illustrated in FIG. 9 only the influential variables selected by the user are used as the axes of the graphs, all the extracted influential variables may be used as the axes of the graphs.

The examples to be presented extracted by the example extraction function 152 are mapped on each of the graphs. A dot 8 on the graph with “tumor marker” and “leukocyte count” as the axes is an example of a patient name “XX” with “leukocyte count” of “2,500/mm³ and a value of “tumor marker” of 8 ng/ml, for example. The graphs illustrated in FIG. 9 are examples of a plane defined by the values of two kinds of examination results.

The output control function 154, when the user selects a dot representing an example with a pointer of a mouse, for example, may display the patient information, the true value, the first diagnostic result, the second diagnostic result, and the diagnostic result by the doctor 4 corresponding to the example.

The output control function 154 causes the display 140 to output the example to be presented with the diagnosis influence degree indicator and the updating influence degree indicator associated with the example to be presented. In the example illustrated in FIG. 9, the output control function 154 displays on the graph the examples to be presented for which the diagnosis influence degree indicator or the updating influence degree indicator is equal to or greater than a threshold in an emphasized manner on the graph. Thus, the user can grasp whether the diagnosis influence degree indicator or the updating influence degree indicator of the examples to be presented mapped on the graph is equal to or greater than the threshold. In the example illustrated in FIG. 9, a dot indicating the example to be presented for which the diagnosis influence degree indicator or the updating influence degree indicator is equal to or greater than the threshold is displayed larger than a dot indicating the example to be presented for which the diagnosis influence degree indicator or the updating influence degree indicator is smaller than the threshold.

The threshold of the diagnosis influence degree indicator and the threshold of the updating influence degree indicator can each be changed by the user through a threshold adjustment unit 90 on the analytical screen. The output control function 154 displays the threshold adjustment unit 90 including an operable knob and a bar. In FIG. 9, to enable the user to understand the details of the respective indicators, the diagnosis influence degree indicator is denoted by “difference between CDSS (before updating) and user diagnostic result,” whereas the updating influence degree indicator is denoted by “difference between CDSS before and after updating and true value.”

Although in the example illustrated in FIG. 9, the example to be presented is displayed in an emphasized manner when both the diagnosis influence degree indicator and the updating influence degree indicator are equal to or greater than thresholds, a condition for displaying in an emphasized manner may be that at least either one of the diagnosis influence degree indicator or the updating influence degree indicator is equal to or greater than the threshold may be a condition for displaying in an emphasized manner.

In the example illustrated in FIG. 9, in the example of the patient name “XX” corresponding to the dot 8, both the diagnosis influence degree indicator and the updating influence degree indicator are equal to or greater than the threshold. That is to say, in the example of the patient name “XX,” even though the influence exerted on the diagnosis by the doctor 4 is as large as being equal to or greater than the threshold, updating causes the diagnostic result to separate from the true value by a value equal to or greater than the threshold compared with that before updating.

The example to be presented associated with the diagnosis influence degree indicator of a value equal to or greater than the threshold set by the user and the updating influence degree indicator of a value equal to or greater than the threshold will be an object for which the accuracy of the diagnosis by the CDSS 20 is preferentially increased in update processing described below.

Referring back to FIG. 1, the reception function 155 receives operations by the user via the input interface 130. The reception function 155 receives an operation by the user adjusting the threshold of the diagnosis influence degree indicator or the threshold of the updating influence degree indicator through the threshold adjustment unit 90 on the analytical screen, for example. In addition, the reception function 155 receives an operation selecting a variable by the user through the list 91 of influential variables on the analytical screen.

The reception function 155 sends out the received operation details to the output control function 154 and the data processing function 156.

The data processing function 156 processes the new learning data by the user based on the threshold of the diagnosis influence degree indicator and the threshold of the updating influence degree indicator set by the user and the degrees of importance of the respective variables calculated by the influential variable extraction function 153.

More specifically, the data processing function 156 includes the generation function 157 and the classification function 158.

The generation function 157 replicates an example for which the diagnosis influence degree indicator is equal to or greater than the threshold among the examples included in the new learning data to generate new examples and adds the new examples to the new learning data.

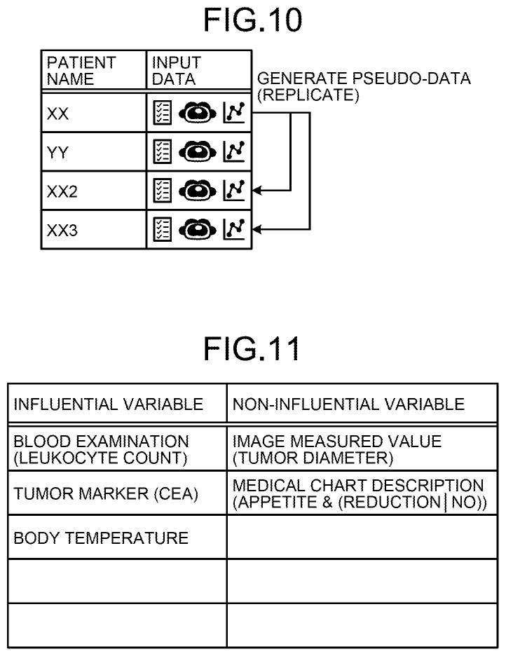

FIG. 10 is a diagram of an example of addition of the new examples according to the first embodiment. As illustrated in FIG. 10, the generation function 157 replicates the example of the patient name “XX” for which both the diagnosis influence degree indicator and the updating influence degree indicator are equal to or greater than the respective thresholds among the examples included in the new learning data to generate examples of a patient name “XX2” and a patient name “XX3.” The examples of the patient name “XX2” and the patient name “XX3” are pieces of pseudo-data, which are not actual diagnostic histories. The examples of the patient name “XX2” and the patient name “XX3” include the same information as that of the example of the patient name “XX” other than identification information such as the patient name.

With such replication, the examples having the same patient information and true value as those of the example of the patient name “XX” increase within the new learning data, and thus in updating of the CDSS 20, learning is performed so as to improve diagnostic accuracy for an example having the patient information similar to that of the patient name “XX.” Replicating an example to increase samples having the same patient information and true value within the new learning data by the generation function 157 is an example of a method for increasing a weight for the example.

The generation function 157, may select not only the example identified by the threshold of the diagnosis influence degree indicator and the threshold of the updating influence degree indicator set by the user, but also automatically select an example to be replicated.

The classification function 158 classifies the variables included in the new learning data into the “influential variable,” which has a large influence exerted on updating of the CDSS 20 and a “non-influential variable,” which has a small influence exerted on updating of the CDSS 20 exerts.

FIG. 11 is a diagram of an example of classification of variables according to the first embodiment. In the example illustrated in FIG. 11, three variables selected by the user on the analytical screen cut of influential variable candidates extracted by the influential variable extraction function 153 are classified into the “influential variable,” whereas the other influential variable candidates are classified into the “non-influential variable.” In addition, variables not having been extracted as the influential variable candidates are also classified into the “non-influential variable.”

The classification function 158 may perform classification into the “influential variable” and the “non-influential variable” based on not selection by the user but the degrees of importance of the respective influential variable candidates. Alternatively, the classification function 158 may classify all the influential variable candidates extracted by the influential variable extraction function 153 into the “influential variable” and classify only variables not having been extracted as the influential variable candidates into the “non-influential variable.”

Referring back to FIG. 1, the learning control function 159, in learning of the CDSS 20, controls learning processing so as to provide an example having a smaller difference between the first diagnostic result and the diagnostic result by the doctor 4 among the examples included in the new learning data with a higher priority.

More specifically, the learning control function 159, in learning of the CDSS 20, controls the learning processing so as to minimize the difference between the second diagnostic result by the CDSS 20 and the true value.

Furthermore, the learning control function 159 classifies the kinds of examination results included in the disease-related information into a first examination result, which has a large influence exerted on a change between the first diagnostic result and the second diagnostic result, and a second examination result, which has a smaller influence exerted on the change between the first diagnostic result and the second diagnostic result than the first examination result has. The learning control function 159 inputs the first examination result to a first model and inputs the second examination result to a second model. The learning control function 159 then determines a weighted average of an output result of the first model and an output result of the second model with a weighting coefficient adjusted so as to increase a weight of the output result of the first model and outputs a calculation result of the weighted average as a prediction result. That is to say, the learning control function 159 adjusts the weighting coefficient to adjust the influence that the “influential variable” exerts on updating. It is assumed that both the first model and the second model are CDSSs.

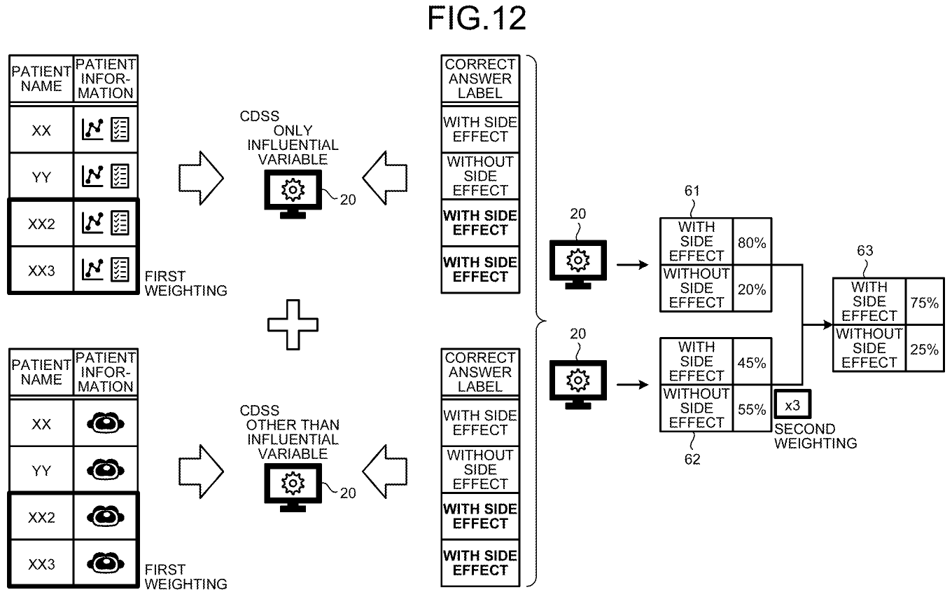

FIG. 12 is a diagram of an example of the learning processing according to the first embodiment. The learning control function 159 updates the CDSS 20 based on processed learning data obtained by processing the new learning data by the data processing function 156. “Correct answer label” illustrated in FIG. 12 indicates the true value.

As illustrated in FIG. 12, the learning control function 159 obtains a first learning result 61, which has learned only the “influential variable” out of the new learning data, and a second learning result 62, which has learned only the “non-influential variable” out of the new learning data. The learning control function 159 further uses a result obtained by multiplying the second learning result 62 by the weighting coefficient and the first learning result 61, which is not multiplied by the weighting coefficient, as learning data to increase the influence of the “non-influential variable and to reduce the influence of the “influential variable” in updating of the CDSS 20. The value of the weighting coefficient indicated in FIG. 12 is an example and is not limited to this example.

As illustrated in FIG. 12, the new examples replicated by the generation function 157 are added in the processed learning data. Thus, the learning control function 159 performs two kinds of weighting, or first weighting by addition of the new examples and second weighting for the “non-influential variable,” to perform learning of the CDSS 20.

The learning control function 159 further performs weighting in an algorithm of the learning processing as well. The weighting is weighting that provides an example where a difference between the first diagnostic result and the diagnostic result by the doctor 4 is small, that is, an example where as the value of the diagnosis influence degree indicator becomes larger, the priority becomes higher.

In the present embodiment, the learning control function 159 performs learning using a minimizing model of a weighted loss function. The learning control function 159 employs the mean square error (MSE), for example. The following shows a model for generally making the second diagnostic result by the CDSS 20 close to the true value by the MSE as Expression (3).

$$M S E = \frac{{\sum }_{i = 1}^{n} {( {y}_{i} - {y}_{i}^{p} )}^{2}}{n} \\ {y}_{i}^{p} \text{:} DIAGNOSTIC RESULT BY CDSS \\ {y}_{i} \text{:} TRUE VALUE \qquad ( 3 )$$

However, the learning control function 159 of the present embodiment performs learning using a minimizing model of Expression (4) with Expression (3) weighted. In Expression (4), the weighting coefficient of the example having a smaller difference between the first diagnostic result and the diagnostic result by the doctor 4 is increased. Thus, in Expression (4), not only simply making the second diagnostic result close to the true value, examples for which the value of the diagnosis influence degree indicator is large exerts higher influence on learning of the CDSS 20. The method of learning is not limited to the MSE, and another method of machine learning may be employed.

$$\frac{{\sum }_{i = 1}^{n} \frac{1}{{( {y}_{i}^{u} - {y}_{i}^{{p}^{′}} )}^{2} + 1} \cdot {( {y}_{i} - {y}_{i}^{p} )}^{2}}{n} \\ {y}_{i}^{{p}^{′}} : DIAGNOSTIC RESULT BY CDSS BEFORE UPDATING \\ {y}_{i}^{p} \text{:} DIAGNOSTIC RESULT BY CDSS AFTER UPDATING \\ {y}_{i}^{u} \text{:} DIAGNOSTIC RESULT BY DOCTOR \\ {y}_{i} \text{:} TRUE VALUE \frac{1}{{( {y}_{i}^{u} - {y}_{i}^{{p}^{′}} )}^{2} + 1} \text{:} WEIGHT \qquad ( 4 )$$

Although in the present embodiment the learning control function 159 of the medical information processing apparatus 10 controls the learning processing of the CDSS 20, the learning control function 159 may be a function of the CDSS 20.

The following describes a procedure of updating support processing of the CDSS 20 executed by the medical information processing apparatus 10 configured as described above.

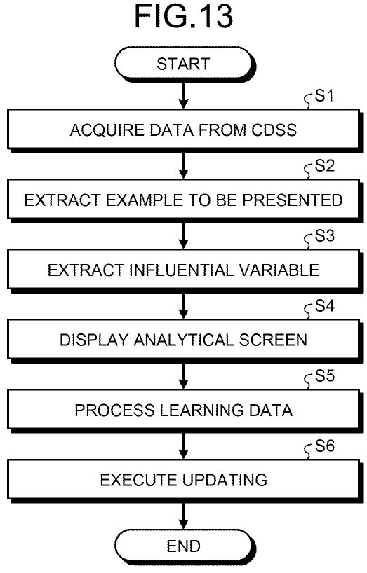

FIG. 13 is a flowchart of an example of the procedure of the updating support processing according to the first embodiment.

First, the acquisition function 151 acquires data for use in the updating support processing from the CDSS 20 (S1). Specifically, the acquisition function 151 acquires the first diagnostic result and the second diagnostic result for each of the patients P having undergone diagnosis, the diagnostic result by the doctor 4 for each of the patients P having undergone diagnosis supported by the CDSS 20 before updating, and the new learning data.

Next, the example extraction function 152 extracts the example that is subjected to the influence of updating of the CDSS 20 by learning of the new learning data by the prescribed criterion or more among the examples included in the new learning data as the example to be presented based on the first diagnostic result, the second diagnostic result, the diagnostic result by the doctor 4 for each of the patients P having undergone diagnosis supported by the CDSS 20 before updating (S2). The example extraction function 152 calculates the updating influence degree indicator and the diagnosis influence degree indicator for each example.

The influential variable extraction function 153 extracts the item having influenced the change in the diagnostic result by the CDSS 20 before and after updating among the kinds of examination results for each of the patients P having undergone diagnosis included in the new learning data as the influential variable (S3).

The output control function 154 causes the display 140 to display the analytical screen representing the example to be presented extracted by the example extraction function 152 in association with the updating influence degree indicator, the diagnosis influence degree indicator, and the influential variable extracted by the influential variable extraction function 153 (S4). The reception function 155 receives various kinds of operations by the user to the analytical screen.

The generation function 157 and the classification function 158 included in the data processing function 156 process the new learning data based on setting of the threshold of the updating influence degree indicator, setting of the threshold of the diagnosis influence degree indicator, and selection of the influential variable by the user (S5). The generation function 157 and the classification function 158 may process the new learning data based on not the operations by the user but a prescribed criterion, a calculation result, or the like.

The learning control function 159 executes updating of the CDSS 20 using the processed learning data (S6). Then, the processing of this flowchart ends.

Thus, the medical information processing apparatus 10 of the present embodiment causes the display 140 to display the example that is subjected to the influence of updating of the CDSS 20 by the first prescribed criterion or more among the examples included in the new learning data in association with the diagnosis influence degree indicator. Thus, the user can easily grasp one having a high degree of the influence exerted on the diagnosis by the doctor 4 among the examples to be presented being subject to the influence of updating of the CDSS 20 by the prescribed criterion or more, and the user can select an example to be prioritized in learning of the CDSS 20. Thus, the medical information processing apparatus 10 of the present embodiment can support to preferentially increase the diagnostic accuracy of a case to which importance is attached during the diagnosis by the doctor 4 in learning of the CDSS 20.

The medical information processing apparatus 10 of the present embodiment causes the display 140 to display the example to be presented with the updating influence degree indicator associated with the example to be presented. Thus, the medical information processing apparatus 10 of the present embodiment enables the user to easily grasp one that is significantly subjected to the influence of updating of the CDSS 20 among the examples to be presented.

The medical information processing apparatus 10 of the present embodiment extracts the example in which the first difference is smaller than the second difference and the difference between the first diagnostic result and the diagnostic result by the doctor 4 supported by the CDSS 20 is the second prescribed criterion or less among the examples included in the new learning data as the example to be presented. Thus, the medical information processing apparatus 10 of the present embodiment enables the user to easily grasp an example for which the doctor 4 has been influenced by the diagnostic result by the CDSS 20 when giving diagnosis.

The diagnosis influence degree indicator of the present embodiment indicates the degree of the influence that the CDSS 20 exerts on the diagnosis by the doctor 4. The diagnosis influence degree indicator has a larger value when the difference between the first diagnostic result and the diagnostic result by the doctor supported by the CDSS 20 is smaller. Thus, the medical information processing apparatus 10 of the present embodiment can easily determine whether an example is the example for which the doctor 4 has been influenced by the diagnostic result by the CDSS 20 when giving diagnosis.

The medical information processing apparatus 10 of the present embodiment replicates the example for which the diagnosis influence degree indicator is equal to or greater than the threshold among the examples included in the new learning data to generate the new examples and adds the new examples to the new learning data. Thus, the medical information processing apparatus 10 of the present embodiment increases the number of samples the diagnosis influence degree indicator of which is equal to or greater than the threshold in the new learning data and can thereby predominantly improve the diagnostic accuracy of the CDSS 20 for an example having a high possibility that the doctor 4 refers to during diagnosis.

The medical information processing apparatus 10 of the present embodiment, in learning of the CDSS 20, controls the learning processing so as to provide the example having a smaller difference between the first diagnostic result and the diagnostic result by the doctor 4 supported by the CDSS 20 among the examples included in the new learning data with a higher priority. Thus, the medical information processing apparatus 10 of the present embodiment can predominantly improve the diagnostic accuracy of the CDSS 20 for the example having a high possibility that the doctor 4 refers to during diagnosis.

The medical information processing apparatus 10 of the present embodiment, in learning of the CDSS 20, controls the learning processing so as to minimize the difference between the second diagnostic result by the CDSS 20 and the true value. Thus, the medical information processing apparatus 10 of the present embodiment can improve the accuracy of the diagnosis by the CDSS 20 by learning.

The medical information processing apparatus 10 of the present embodiment controls the learning processing so as to reduce the priority of one exerting an influence on the change between the first diagnostic result and the second diagnostic result among the kinds of examination results included in the new learning data. Thus, the medical information processing apparatus 10 of the present embodiment reduces a significant change between the diagnostic results by updating of the CDSS 20.

The medical information processing apparatus 10 of the present embodiment extracts the item related to the difference between the first diagnostic result and the second diagnostic result among the items of the kinds of examination results included in the new learning data as the influential variable, which exerts an influence on updating of the CDSS 20, and causes the display 140 to display the example to be presented in association with the influential variable. Thus, the medical information processing apparatus 10 of the present embodiment can use the item related to the difference between the first diagnostic result and the second diagnostic result as an analysis axis of the example to be presented.

Although the present embodiment describes the influential variable extraction function 153 and the classification function 158 as different functions, the influential variable extraction function 153 and the classification function 158 may be configured as one function. The influential variable extraction function 153 extracts an examination item having a high degree of importance as the influential variable having a high degree of importance for updating of the CDSS 20, for example. The influential variable extraction function 153 extracts the influential variable having a high degree of importance based on a variable degree of importance output from a prediction model learned with a label indicating division of “important” or “non-important” for each examination item as a correct answer. An example in which the diagnostic result by the CDSS 20 before updating is close to the diagnostic result by the doctor 4, or having a high diagnosis influence degree, and the difference between the diagnostic result by the CDSS 20 before updating and the diagnostic result by the CDSS 20 after updating is large, or having a large updating influence degree, is determined to be important, whereas an example not being so is determined to be non-important. More specifically, the important examination item is an examination item on an example in which the difference between the diagnostic result by the CDSS 20 before updating and the diagnostic result by the doctor 4 supported by the CDSS 20 is the second prescribed criterion or less and the difference between the diagnostic result by the CDSS 20 before updating and the diagnostic result by the CDSS 20 after updating is a fourth prescribed criterion or more. The fourth prescribed criterion may be a value or another condition.

### Second Embodiment

In the first embodiment described above, the medical information processing apparatus 10 displays examples on the patients P having undergone diagnosis on the analytical screen. In this second embodiment, the medical information processing apparatus 10 also displays examples of patients not having undergone diagnosis on the analytical screen.

The information processing system S of the present embodiment includes the medical information processing apparatus 10 and the CDSS 20 like the first embodiment.

The medical information processing apparatus 10 of the present embodiment includes the NW interface 110, the storage 120, the input interface 130, the display 140, and the processing circuit 150 like the first embodiment.

The processing circuit 150 of the present embodiment includes the acquisition function 151, the example extraction function 152, the influential variable extraction function 153, the output control function 154, the reception function 155, the data processing function 156, the generation function 157, the classification function 158, and the learning control function 159 like the first embodiment. The example extraction function 152, the influential variable extraction function 153, the reception function 155, the data processing function 156, the generation function 157, the classification function 158, and the learning control function 159 include the same functions as those of the first embodiment.

The acquisition function 151 of the present embodiment acquires information on patients not having undergone diagnosis including a plurality of examples not having undergone diagnosis on disease-related information for each of a plurality of patients not having undergone diagnosis by the doctor 4 and the CDSS 20 in addition to the function of the first embodiment. An origin from which the information on patients not having undergone diagnosis is acquired is an electronic medical record system, for example.

The information on patients not having undergone diagnosis is an example of second data of the present embodiment. The information on patients not having undergone diagnosis includes the disease-related information for each of the patients not having undergone diagnosis. In the information on patients not having undergone diagnosis, the disease-related information associated with one patient is one example not having undergone diagnosis, for example. The information on patients not having undergone diagnosis includes a plurality of examples not having undergone diagnosis.

The disease-related information included in the information on patients not having undergone diagnosis includes a plurality of kinds of examination results, that is, variables for each of the patients not having undergone diagnosis.

The output control function 154 of the present embodiment causes the display 140 to display information representing a possibility of the diagnostic result by the CDSS 20 for the patients included in the information on patients not having undergone diagnosis changing before and after learning of the new learning data in addition to the function of the first embodiment.

The information representing the possibility of the diagnostic result by the CDSS 20 for the patients included in the information on patients not having undergone diagnosis changing before and after learning of the new learning data is information indicating whether the examples not having undergone diagnosis included in the information on patients not having undergone diagnosis correspond to a variable for which the difference in the diagnostic result by the CDSS 20 before and after updating is large, for example.

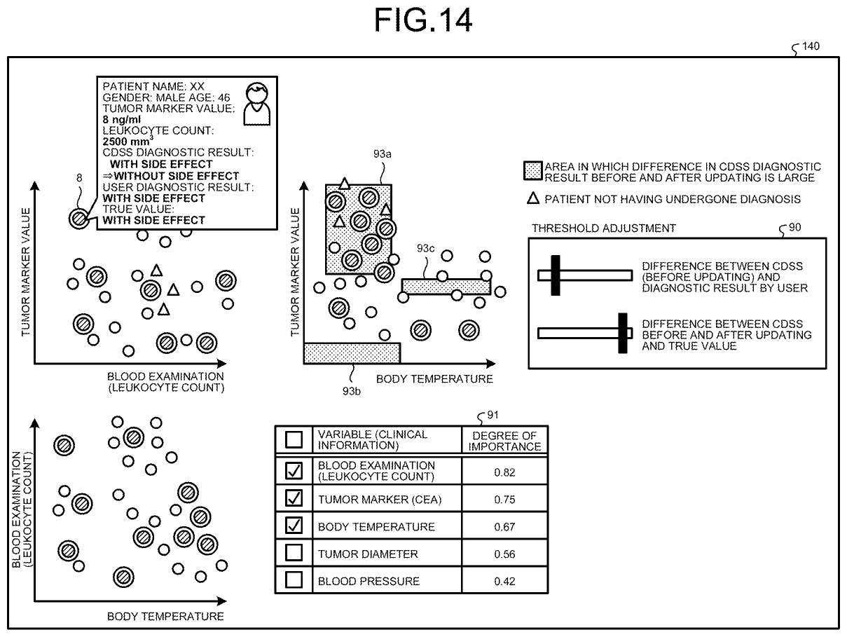

FIG. 14 is a diagram of an example of the analytical screen according to the second embodiment. In the example illustrated in FIG. 14, the output control function 154 displays areas 93a to 93c (hereinafter, referred to simply as an area 93) for which the difference in the diagnostic result by the CDSS 20 before and after updating of the CDSS 20 is large on the graph. That the difference in the diagnostic result by the CDSS 20 before and after updating of the CDSS 20 is large means that the difference between the first diagnostic result and the second diagnostic result is the fourth prescribed criterion or more. As described above, the fourth prescribed criterion may be a value or another condition. In the example illustrated in FIG. 14, a range in which the difference between the first diagnostic result and the second diagnostic result of the patient P having undergone diagnosis is the fourth prescribed criterion or more on a graph with a variable “tumor marker value” as the vertical axis and with a variable “body temperature” as the horizontal axis is the area 93.

As in the graph illustrated in FIG. 14, the output control function 154 displays the examples of the patients P having undergone diagnosis included in the new learning data and the undiagnosed examples on the patients not having undergone diagnosis included in the information on patients not having undergone diagnosis in a mapped manner on the graph defined by two kinds of variables. The graph of the analytical screen illustrated in FIG. 14 is an example of the plane defined by the values of two kinds of examination results.

Thus, when an undiagnosed example mapped on the graph is present within the area 93, there is a high possibility that the diagnostic result by the CDSS 20 for the patient of the example not having undergone diagnosis will change before and after learning of the new learning data. When many undiagnosed examples are present within the area 93, it is desirable that the user carefully perform selection of the threshold and the like so that the accuracy of the diagnosis for the patients corresponding to these examples not having undergone diagnosis does not reduce by updating of the CDSS 20.

Thus, the medical information processing apparatus 10 of the present embodiment acquires the information on patients not having undergone diagnosis and causes the display 140 to display the information representing the possibility of the diagnostic result by the CDSS 20 for the patients included in the information on patients not having undergone diagnosis changing before and after learning of the new learning data. Thus, the medical information processing apparatus 10 of the present embodiment also enables the user to easily grasp an influence to which the diagnosis for the patients not having undergone diagnosis is subjected by updating of the CDSS 20 in addition to having the effect of the first embodiment. By using the medical information processing apparatus 10 of the present embodiment, the user can, before updating of the CDSS 20, grasp to what extent the updating influences diagnosis for patients slated to undergo diagnosis in the future, for example.

The medical information processing apparatus 10 of the present embodiment displays the examples of the patients P having undergone diagnosis included in the new learning data and the undiagnosed examples on the patients not having undergone diagnosis included in the information on patients not having undergone diagnosis in a mapped manner on the graph defined by two kinds of variables. Thus, the medical information processing apparatus 10 of the present embodiment enables the user to visually grasp the distribution of the examples of the patients having undergone diagnosis and the distribution of the examples of the patients not having undergone diagnosis.

First Modification

Although the analytical screen of the embodiments described above displays a simulation result when updating of the CDSS 20 is performed on one condition, simulation results on a plurality of different conditions may be displayed on one screen.

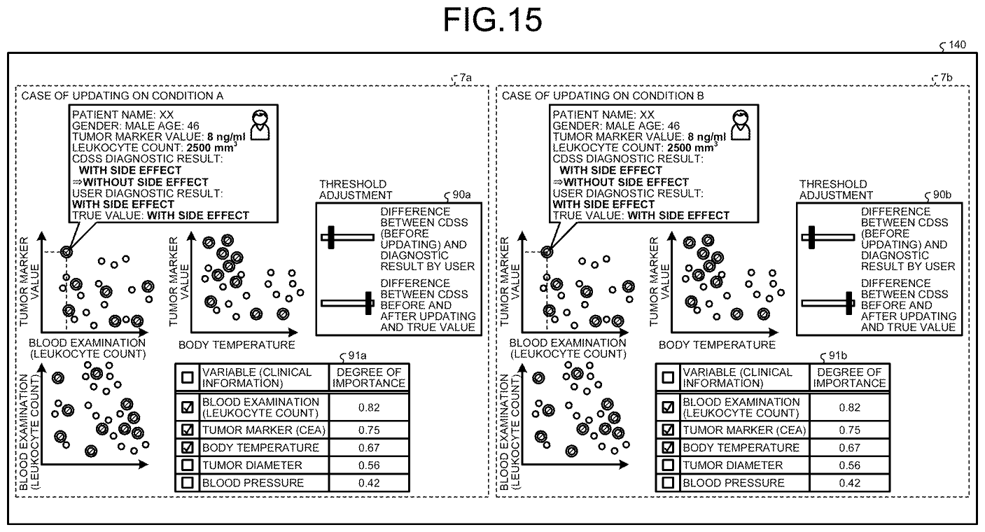

FIG. 15 is a diagram of an example of the analytical screen according to the first modification. As illustrated in FIG. 15, the output control function 154 of the present modification outputs examples to be presented when the CDSS 20 is updated on a plurality of different conditions on the same screen for each of the different conditions.

A first screen area 7a and a second screen area 7b of the analytical screen illustrated in FIG. 15 represent the examples to be presented in the CDSS 20 updated on the respective different conditions. The details displayed within each of the screen areas are the same as those of the analytical screen of the first embodiment illustrated in FIG. 9 or those of the analytical screen of the second embodiment illustrated in FIG. 14. The first screen area 7a and the second screen area 7b include threshold adjustment units 90a and 90b, respectively, lists 91a and 91b of influential variables, respectively, and graphs with selected variables as the axes, for example.

Examples of the different conditions include, but are not limited to, being different in the contents of the new learning data, being different in an algorithm of the CDSS 20 to be updated, and being different in the past learning data used in updating before updating by the new learning data.

Thus, the simulation results on the different conditions are displayed on one screen, thereby enabling the user to easily consider about which condition should be employed by comparing them with each other.

Second Modification

In the analytical screen of the embodiments described above, the diagnosis influence degree indicator is set by the difference between the first diagnostic result and the diagnostic result by the doctor 4 supported by the CDSS 20. However, the method for determining the magnitude of the influence that the CDSS 20 exerts on the diagnosis by the doctor 4 is not limited to this example.

For the magnitude of the influence that the CDSS 20 exerts on the diagnosis by the doctor 4, self-declaration by the doctor 4 may be employed, for example. The diagnosis influence degree indicator of the present modification is a declaration result by the doctor 4 who has performed diagnosis on the patient P as the example to be presented on an extent of being subjected to an influence from the CDSS 20 during diagnosis. The declaration result is a result obtained by declaring whether the doctor 4 has been influenced by the diagnostic result by the CDSS 20 during diagnosis by the doctor 4 himself/herself. The declaration result may be not only the occurrence of the influence but also information representing the magnitude of the degree of being influenced with a value, a level, or the like. The acquisition function 151 of the present modification acquires the declaration result for each example from an electronic medical record system, for example. Alternatively, the reception function 155 may receive input of the declaration result for each example by the doctor 4.

Third Modification

The configuration of the information processing system S is not limited to the example described above. Although the embodiments described above describe the CDSS 20 and the medical information processing apparatus 10 as separate apparatuses, for example, the CDSS 20 and the medical information processing apparatus 10 may be integrated with each other as one apparatus. In this case, the CDSS 20 may be an example of the medical information processing apparatus.

The information processing system S may include a plurality of CDSSs 20. Part or the whole of the CDSS 20 and the medical information processing apparatus 10 may be implemented in a cloud environment.

Fourth Modification

The learned models of the embodiments described above include a “self-learning model,” in which the user gives feedback to a result output by the learned models to further update an internal algorithm of the learned models.

In the embodiments described above, the processing described with the learned models as an example of the method of implementation may be implemented by a method other than machine learning or deep learning.

Fifth Modification

In the embodiments described above, the user adjusts the threshold of the diagnosis influence degree indicator or the threshold of the updating influence degree indicator through the threshold adjustment unit 90 on the analytical screen to indirectly select the example to be prioritized in updating of the CDSS 20. The method of selection is not limited to this example; the user may directly select the example to be prioritized in updating of the CDSS 20 by selecting the dot S displayed on the graph on the analytical screen.

At least one embodiment described above can support to preferentially increase the diagnostic accuracy of a case to which importance is attached during diagnosis by a doctor in learning of a clinical decision support system.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry, the processing circuitry being configured to
acquire a first diagnostic result for each of a plurality of patients having undergone diagnosis by a clinical decision support system (CDSS) before learning of first data, a second diagnostic result for each of the patients having undergone diagnosis by the clinical decision support system after learning of the first data, a diagnostic result by a doctor for each of the patients having undergone diagnosis, and the first data,
extract an example that is subjected to an influence of updating of the clinical decision support system by learning of the first data by a first prescribed criterion or more among a plurality of examples included in the first data as an example to be presented based on the first diagnostic result, the second diagnostic result, and the diagnostic result by the doctor, and
output the example to be presented in association with a diagnosis influence degree indicator indicating a degree of an influence that the clinical decision support system exerts on diagnosis by the doctor in the example to be presented,
the first data including the examples in which disease-related information for each of the patients having undergone diagnosis and a true value representing a treatment result for each of the patients having undergone diagnosis are associated with each other.

2. The medical information processing apparatus according to claim 1, wherein the processing circuitry is configured to output the example to be presented with an updating influence degree indicator representing magnitude of a difference between a first difference as a difference between the first diagnostic result and the true value, and a second difference as a difference between the second diagnostic result and the true value associated with the example to be presented.

3. The medical information processing apparatus according to claim 2, wherein the processing circuitry is configured to extract an example in which the first difference is smaller than the second difference and a difference between the first diagnostic result and the diagnostic result by the doctor supported by the clinical decision support system is a second prescribed criterion or less among the examples included in the first data as the example to be presented.

4. The medical information processing apparatus according to claim 1, wherein a value of the diagnosis influence degree indicator becomes larger as a difference between the first diagnostic result and the diagnostic result by the doctor supported by the clinical decision support system becomes smaller.

5. The medical information processing apparatus according to claim 1, wherein the processing circuitry is configured to
replicate an example for which the diagnosis influence degree indicator is equal to or greater than a threshold among the examples included in the first data to generate a new example and
add the new example to the first data.

6. The medical information processing apparatus according to claim 1, wherein the processing circuitry is configured to
learn so as to provide an example having a smaller difference between the first diagnostic result and the diagnostic result by the doctor supported by the clinical decision support system among the examples included in the first data with a higher priority, in learning of the clinical decision support system.

7. The medical information processing apparatus according to claim 6, wherein the processing circuitry is configured to
learn so as to minimize a difference between the second diagnostic result by the clinical decision support system and the true value, in learning of the clinical decision support system.

8. The medical information processing apparatus according to claim 6, wherein
the disease-related information included in the first data includes a plurality of kinds of examination result for each of the patients having undergone diagnosis, and
the processing circuitry is configured to
classify the kinds of examination results into a first examination result, which has a large influence exerted on a change between the first diagnostic result and the second diagnostic result, and a second examination result, which has a smaller influence exerted on the change between the first diagnostic result and the second diagnostic result than the first examination result has,
input the first examination result to a first model,
input the second examination result to a second model, and
determine a weighted average of an output result of the first model and an output result of the second model with a weighting coefficient adjusted so as to increase a weight of the output result of the first model, and output a calculation result of the weighted average as a prediction result.

9. The medical information processing apparatus according to claim 1, wherein the processing circuitry is configured to
determine that when a degree of similarity between the first diagnostic result and the diagnostic result by the doctor supported by the clinical decision support system is higher, the influence is larger, and
extract an example having an influence exerted on the diagnostic result by the doctor larger than a third prescribed criterion among the examples included in the first data as the example to be presented.

10. The medical information processing apparatus according to claim 1, wherein
the processing circuitry is configured to
extract, based on a variable degree of importance output from a prediction model learned with a label indicating division of change example or no change example for items of a plurality of kinds of examination results included in the disease-related information as a correct answer, an examination item having a high degree of importance among the items of the kinds of examination results as an influential variable having a high degree of importance for updating of the clinical decision support system and
output the example to be presented in association with the influential variable,
each of the plurality of examples included in the first data is identified as the change example or the no change example based on a difference between the first diagnostic result and the diagnostic result by the doctor supported by the medical decision support system and a difference between the first diagnostic result and the second diagnostic result.

11. The medical information processing apparatus according to claim 1, wherein the processing circuitry is configured to
acquire second data including disease-related information for each of a plurality of patients not having undergone diagnosis by the doctor or the clinical decision support system and
output information representing a possibility of a diagnostic result by the clinical decision support system for the patients included in the second data changing before and after learning of the first data.

12. The medical information processing apparatus according to claim 11, wherein
the disease-related information included in the first data or the second data includes a plurality of kinds of examination results for each of the patients having undergone diagnosis or each of the patients not having undergone diagnosis, and
the processing circuitry is configured to cause a display to display the examples included in the first data and the examples on the patients not having undergone diagnosis included in the second data in a mapped manner on a plane defined by values of two kinds of examination results among the kinds examination results.

13. The medical information processing apparatus according to claim 1, wherein the processing circuitry is configured to output the example to be presented when the clinical decision support system has been updated on a plurality of different conditions on the same screen for each of the different conditions.

14. The medical information processing apparatus according to claim 1, wherein the diagnosis influence degree indicator is a declaration result by the doctor who has performed diagnosis on the patient as the example to be presented on an extent of being subjected to an influence from the clinical decision support system during diagnosis.

15. A medical information processing apparatus comprising processing circuitry, the processing circuitry being configured to
acquire first data for use in learning of a clinical decision support system and second data including disease-related information for each of a plurality of patients not having undergone diagnosis by a doctor or the clinical decision support system and
output information representing a possibility of a diagnostic result by the clinical decision support system for the patients included in the second data changing before and after learning of the first data.
